(19) United States
(12) Patent Application Publication
(10) Pub. No.: US 2026/0289992 A1
(43) Pub. Date: Sep. 24, 2026

(54) TACTILE SENSOR AND CAMERA-BASED APPARATUS AND METHOD FOR BODY CENTER OF PRESSURE TRAJECTORY ESTIMATION USING AN ARTIFICIAL NEURAL NETWORK

(71) Applicant: GWANGJU INSTITUTE OF SCIENCE AND TECHNOLOGY, Gwangju (KR)

(72) Inventors: Hosu LEE, Jinju-si (KR); Kyung-Joong KIM, Gwangju (KR); Yun Ho CHOI, Gwangju (KR); Jaehyeon BAIK, Busan (KR)

(73) Assignee: GWANGJU INSTITUTE OF SCIENCE AND TECHNOLOGY, Gwangju (KR)

(21) Appl. No.: 19/575,255

(22) Filed: Mar. 23, 2026

(30) Foreign Application Priority Data
Mar. 24, 2025 (KR) 10-2025-0037300

(51) Int. Cl. G06V 10/82; G01L 1/18; G01L 1/20; G06F 3/045; G06V 10/44; G06V 10/62; G06V 20/40; G06V 40/10; G06V 40/20; G06V 40/70

(52) U.S. Cl. CPC G06V 10/82; G01L 1/18; G01L 1/205; G06F 3/045; G06V 10/454; G06V 10/62; G06V 20/46; G06V 40/103; G06V 40/23; G06V 40/70; G06V 2201/12

(57) ABSTRACT

An apparatus for estimating a trajectory of a body center of pressure using an artificial neural network, including: a pressure data acquisition unit configured to acquire, over time, pressure image data of a sole based on point-by-point pressure data of a plate-type tactile sensor; a posture data acquisition unit configured to acquire, over time, three-dimensional (3D) body keypoint data based on two-dimensional (2D) skeleton data extracted from images of a plurality of RGB cameras; and an estimation unit configured to estimate, at each sequence length, two-dimensional (2D) coordinates of the body center of pressure by sequentially inputting, to a pre-trained deep learning model, pressure image data and 3D body keypoint data in units of a preset (predetermined) sequence length.

### CROSS-REFERENCE TO RELATED APPLICATION

This application claims priority to Korean Patent Application No. 10-2025-0037300 filed with the Korean Patent Office on Mar. 24, 2025, the entire contents of each of which are incorporated herein by reference for all purposes.

### FIELD

The present disclosure relates to a tactile sensor and camera-based body center of pressure (CoP) trajectory estimation apparatus and a method thereof using an artificial neural network, and more particularly, to a tactile sensor and camera-based body center of pressure (CoP) trajectory estimation apparatus and a method thereof capable of estimating, in real time, a change in a center of pressure of a body by inputting sensing data obtained by a tactile sensor and body keypoint data obtained by an RGB camera to an artificial intelligence model.

The present disclosure was made with the support of the 2025 Gwangju Institute of Science and Technology (GIST)-MIT Joint Research Program (Project No. GK17020).

### BACKGROUND

According to the World Health Organization (WHO)'s “Ageing and Health” report, it is expected that, by 2030, one-sixth of the world's population will be aged 60 or older, and, by 2050, that number is expected to reach 2.1 billion.

This is a figure that has increased very rapidly from the value of 1 billion in 2020, and through this, it can be seen that the elderly population is rapidly increasing worldwide.

In the case of Korea, according to Statistics Korea's “Population Projections for the Future: 2022-2072,” the proportion of the elderly population is expected to exceed 20% in 2025, 30% in 2036, and 40% in 2050, and it can be seen that Korea is also rapidly entering a super-aged society.

According to the World Health Organization (WHO), approximately 684,000 people worldwide die each year due to falls, which is the second-highest number among causes of death due to unintentional injuries.

In particular, death due to falls is most likely to occur in elderly people aged 60 years or older.

Approximately 37.3 million falls occur each year, and many of them lead to injuries requiring medical treatment.

Falls are one of the major causes of accidents in elderly people, and closely related thereto is a deterioration in a sense of balance.

The center of pressure (Center of Pressure, CoP) represents a center of pressure applied to a sole, and moves in real time whenever a body moves.

In general, when the center of pressure is within a bound of support (BoS), the body may stably maintain balance.

However, when the center of pressure (CoP) deviates from a base of support (BoS), a likelihood of losing balance may increase.

As such, the center of pressure (CoP) plays a key role in maintaining balance of a body.

Although various technologies for measuring a center of pressure (CoP) have been previously disclosed, most measure only the CoP of a foot (sole), or, even when a body CoP is estimated, estimation is performed by selecting important feature points, and thus it is difficult to accurately estimate the body CoP.

In addition, a technique using an inertial measurement unit (IMU) has a disadvantage in that frequent calibration is required due to drift occurring over time and sensitivity to an external environment such as a magnetic field.

FIG. 1 is a diagram that describes, in detail, a difference between a center of pressure of a foot and a center of pressure of a body.

As illustrated in FIG. 1, a foot plantar center of pressure (Foot Plantar CoP) represents a center of pressure of a load distribution of each foot, and requires a separate insole pressure sensor (Insole Pressure Sensor); although movement of the foot or a load change can be determined, there is a limitation in analysis of a center of pressure and balance of the entire body.

In addition, a technique using the same is mostly focused only on evaluating a sole pressure distribution and balance (e.g., diagnosing a foot disease (plantar fasciitis, flat feet) and gait analysis, etc.).

In contrast, a center of pressure (Body CoP) of a body represents an overall center of pressure of the body obtained by integrating centers of pressure (CoPs) of both feet, and, when the same is utilized, has an advantage in that whole-body balance, a fall-risk assessment, posture correction, and dynamic motion analysis (e.g., rehabilitation training, sports performance analysis, posture correction, etc.) can be performed.

However, there is a drawback in that, in the related art, an expensive force plate is required to measure a body center of pressure (CoP).

Accordingly, there is a need for a system capable of measuring a center of pressure (Body CoP) of a body with high accuracy, having high applicability scalability, mobility, and user accessibility, and being implementable at low cost.

The background art of the present disclosure is disclosed in Korean Laid-Open Patent Publication No. 10-2024-0122252 (published on Aug. 12, 2024).

### SUMMARY

### Technical Problem

The present disclosure is intended to provide a tactile sensor and camera-based body center of pressure (CoP) trajectory estimation apparatus using an artificial neural network, and a method thereof, capable of estimating, over time, a change in a center of pressure of a body by inputting, to an artificial intelligence model, sensing data obtained by a tactile sensor and body keypoint data obtained by an RGB camera.

### Technical Solution

The present disclosure relates to an apparatus for estimating a trajectory of a body center of pressure using an artificial neural network, comprising: a pressure data acquisition unit configured to acquire, over time, pressure image data of a sole based on point-by-point pressure data of a plate-type tactile sensor; a posture data acquisition unit configured to acquire, over time, three-dimensional (3D) body keypoint data based on two-dimensional (2D) skeleton data extracted from images of a plurality of RGB cameras; and an estimation unit configured to estimate, at each sequence length, two-dimensional (2D) coordinates of the center of pressure of the body by sequentially inputting, to a pre-trained deep learning model, pressure image data and 3D body keypoint data in units of a preset (predetermined) sequence length.

In addition, the estimation unit may provide, over time, a center-of-pressure trajectory of a body based on a center of pressure of the body derived for each sequence-length unit.

In addition, the estimation unit may estimate the center of pressure of the body by additionally inputting, to the deep learning model, body information including at least one of a height, a sex, a weight, a lean body mass, an upper limb length, a lower limb length, and a waist-to-hip ratio of a subject.

In addition, the deep learning model may be implemented to include a CNN-Bi-LSTM model having a structure in which a convolutional neural network (CNN) encoder configured to apply a sliding window to the pressure image data having the preset (predetermined) sequence length to extract a spatial feature within the window and a bidirectional long short-term memory (Bi-LSTM) decoder configured to bidirectionally learn a temporal dependency with respect to the spatial feature are combined.

In addition, the tactile sensor may measure point-by-point pressure through resistance changes generated at respective intersection points between a plurality of electrode lines orthogonally arranged in a grid pattern on both surfaces of a plate-shaped piezoresistive film.

In addition, the pressure data acquisition unit may acquire N×M grayscale pressure image data in which numerical values, obtained by normalizing, to values within a set range, pressure values measured for each of intersection points between a plurality of electrode lines orthogonally arranged in N rows and M columns in the tactile sensor, are used as pixel values on respective two-dimensional coordinates.

In addition, the posture data acquisition unit can acquire, as the three-dimensional (3D) body keypoint data, 3D body keypoint coordinates of a 3D skeleton based on a result of reconstructing, into the 3D skeleton through a triangulation technique, 2D body keypoint coordinates of a 2D skeleton extracted from images of two RGB cameras.

In addition, the present disclosure includes, in a body center of pressure (CoP) trajectory estimation method performed by a body center of pressure (CoP) trajectory estimation apparatus using an artificial neural network, acquiring, over time, pressure image data of a sole based on point-by-point pressure data of a plate-type tactile sensor; acquiring, over time, three-dimensional (3D) body keypoint data based on two-dimensional (2D) skeleton data extracted from images of a plurality of RGB cameras; and estimating, at each sequence length, two-dimensional (2D) coordinates of a center of pressure of a body by sequentially inputting, to a pre-trained deep learning model, pressure image data and 3D body keypoint data in units of a preset (predetermined) sequence length.

In addition, the body center of pressure (CoP) trajectory estimation method may further comprise providing, over time, a center-of-pressure trajectory of a body based on a center of pressure of the body derived for each sequence-length unit.

### Advantageous Effect

According to the present disclosure, accurate estimation of a center of pressure (CoP) of a body is possible by inputting, to a deep learning model, a high-resolution pressure image acquirable through a tactile sensor without an expensive force plate and three-dimensional posture information acquirable through a low-cost RGB camera.

In addition, the present disclosure can accurately estimate, in real time, a body center of pressure (CoP) trajectory by overcoming a limitation of a conventional tactile sensor capable of providing only vertical-axis tactile information and by integrally using, as an input of a deep learning model, three-dimensional (3D) body keypoint data based on a 3D skeleton.

In addition, the present disclosure may be implemented as a sensor-based system, and thus may be easily installed and used not only in a hospital but also in a home and a rehabilitation center, and may provide real-time data processing and visualization.

The technical effects of the present disclosure (e.g., improved accuracy, real-time estimation, low-cost implementation, ease of installation, etc.) may vary depending on input data quality, sensor resolution, camera arrangement, model structure, and computational resources, and are not to be construed as being fixed to specific numerical performance or specific experimental conditions.

In addition, in some embodiments, different designs may be possible depending on an objective, such as prioritizing accuracy over real-time performance, or conversely applying model lightweighting to minimize latency.

Accordingly, the present disclosure encompasses various implementations for achieving the technical objective of “estimating a body center of pressure (CoP) even without a force plate.”

### BRIEF DESCRIPTION OF DRAWINGS

FIG. 1 is a diagram illustrating in detail a difference between a center of pressure of a foot and a center of pressure of a body.

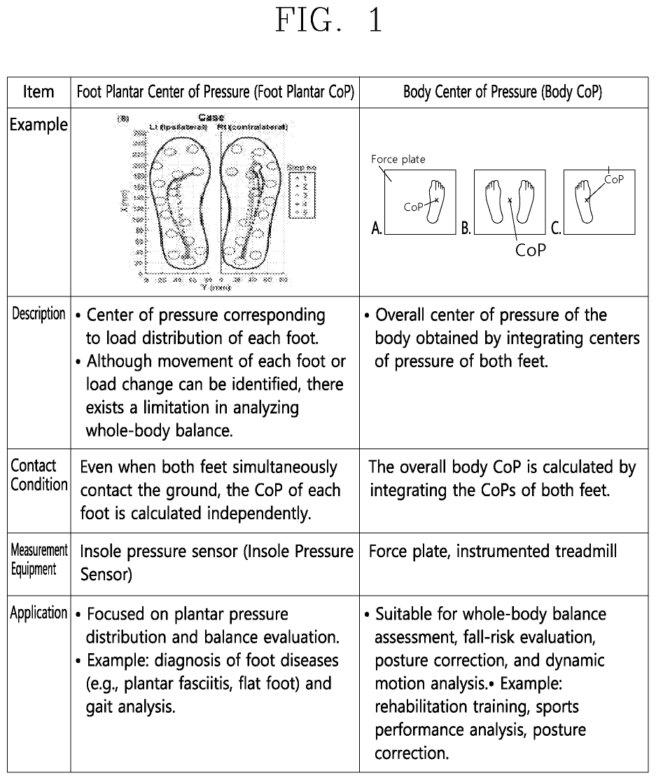

FIG. 2 is a diagram illustrating a configuration of a body center of pressure (CoP) trajectory estimation apparatus using an artificial neural network according to an embodiment of the present disclosure.

FIG. 3 is a diagram illustrating a body center of pressure (CoP) trajectory estimation method using an artificial neural network according to an embodiment of the present disclosure.

FIG. 4 is a diagram illustrating an example of a tactile sensor for an embodiment of the present disclosure.

FIG. 5 is an exemplary diagram visualizing a pressure image and a body center of pressure (CoP) point.

FIG. 6 is a diagram illustrating a principle of extracting a 3D skeleton of a subject by using two RGB cameras in an embodiment of the present disclosure.

FIG. 7 is a view illustrating, by way of example, input and output data of a deep learning model for estimating a center of pressure of a body according to an embodiment of the present disclosure.

FIG. 8 is a diagram illustrating an example of a training and estimation system setting of a deep learning model.

FIG. 9 is a view illustrating a structure of a CNN-Bi-LSTM model according to an embodiment of the present disclosure.

FIG. 10 is a diagram illustrating an experimental environment and a protocol for a performance test of a technique of the present disclosure.

FIG. 11 is a drawing illustrating body CoP estimation performance through a CNN-Bi-LSTM model.

### DETAILED DESCRIPTION OF EMBODIMENTS

Then, with reference to the accompanying drawings, embodiments of the present disclosure will be described in detail so that a person having ordinary skill in the technical field to which the present disclosure pertains can readily carry out the present disclosure.

However, the present disclosure may be embodied in many different forms and is not limited to the embodiments described herein.

In addition, in order to clearly describe the present disclosure in the drawings, portions unrelated to the description are omitted, and throughout the entire specification, similar reference numerals are assigned to similar portions.

Throughout the specification, when a part is described as being “connected” to another part, this includes not only a case where the part is “directly connected,” but also a case where the part is “electrically connected” with another element interposed therebetween.

In addition, when a certain part is said to “include” a certain component, this means, unless specifically stated to the contrary, that other components are not excluded and that other components may be further included.

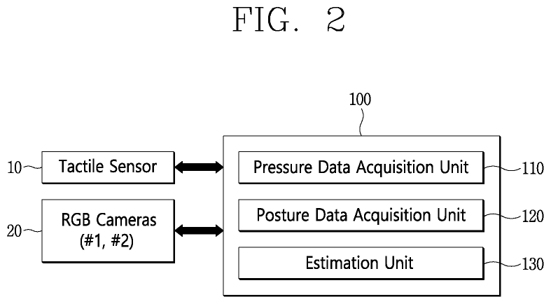

FIG. 2 is a diagram illustrating a configuration of a body center of pressure (CoP) trajectory estimation apparatus according to an embodiment of the present disclosure.

As illustrated in FIG. 2, a body center of pressure (CoP) trajectory estimation apparatus (100) according to an embodiment of the present disclosure includes a pressure data acquisition unit (110), a posture data acquisition unit (120), and an estimation unit (130).

Here, an operation of each unit and a data flow between the units may be performed by a control unit (not shown) corresponding to a processor.

In the present disclosure, “pressure data acquisition unit,” “posture data acquisition unit,” and “estimation unit” exemplify a combination of essential components, and, depending on an implementation purpose (real-time performance, accuracy, cost, ease of installation, etc.), some components may be optionally included or may be replaced.

For example, pressure data may be provided in any form, such as raw pressure values directly acquired from a tactile sensor, a normalized pressure matrix, or a pressure image obtained by visualizing the same, and posture data may also be represented as three-dimensional (3D) body keypoint coordinates, a skeleton representation including the same, or joint angles/segment lengths/center-of-gravity estimates derived from keypoints.

In addition, synchronization of the respective data may be performed in various ways, such as interpolation, resampling, and timestamp-based alignment at the same sampling period or at different periods, and this does not limit the scope of the present disclosure.

The body center of pressure (CoP) trajectory estimation apparatus (100) may operate while being connected, via a network, to a tactile sensor (hereinafter, referred to as a tactile sensor) (10) for measuring pressure data of a sole and a plurality of RGB cameras (20), and may be implemented to include the tactile sensor (10) and the plurality of RGB cameras (20).

A plurality of RGB cameras (20) is exemplified as being configured as two cameras; however, the number of cameras may be expanded to one camera or three or more cameras in order to reduce occlusion of a subject, secure a field of view, and optimize according to an installation environment, and an arrangement (front/side/diagonal) of the cameras, a shooting distance, an angle of view, and a resolution may be changed according to system requirements.

In addition, a RGB-D camera, a stereo camera, or a multi-view image-based pose estimation apparatus, etc. may be used as a replacement for the RGB camera to generate three-dimensional posture information, and such replacement is included in the scope of the present disclosure from a functional standpoint of acquiring “three-dimensional (3D) body keypoint data.”

In addition, the body center of pressure (CoP) trajectory estimation apparatus (100) may be physically configured and implemented as a computer device including a processor, a memory, a user interface input/output device, a storage device, and a network input/output unit, and the body center of pressure (CoP) trajectory estimation apparatus (100) may be implemented as an application program executed on a computer device or a user terminal.

The deep-learning estimation and trajectory providing functions of the estimation unit 130 may be integrally performed in a single apparatus, or may be performed in a distributed manner by a sensor/camera terminal and a computing device (e.g., an edge computing device, a server, or a cloud).

For example, a structure is also possible in which the data acquisition unit operates locally, and the estimation unit utilizes remote computing resources to return only an estimation result to a terminal.

In addition, the estimation unit includes a specific data processing pipeline that performs numerical operations on an input sequence with reference to model parameters in a memory and generates and outputs, as a result thereof, coordinates and a trajectory of a body center of pressure, and thus may be implemented as technical processing that is not limited to simple abstract information processing and that combines sensor-based physical quantities (pressure distribution) and image-based posture information.

The pressure data acquisition unit (110) may acquire, in real time, pressure image data of a sole based on point-by-point pressure data of a plate-type tactile sensor (10) on which a subject's foot is placed, and may provide the acquired pressure image data to the estimation unit (130).

Generation of the pressure image data is exemplified as a grayscale (0 to 255) conversion; however, the generation may be replaced with various preprocessing depending on a sensor resolution (N×M), a pressure range, noise characteristics, and a subsequent model input format.

For example, in addition to Min-Max scaling, the normalizing may be performed by applying z-score standardization, clipping, gamma correction, histogram equalization, or log transformation of pressure values, and the pressure image may be configured not only as a single channel but also as multiple channels (e.g., a temporal-difference channel, a foot/left-right separation channel, and a reliability channel).

In addition, it is also possible to use a pressure matrix itself as an input without visualizing pressure values into an image, or to calculate feature quantities (e.g., a foot contact area, a left-right load ratio, and a front-rear load center) and include the calculated feature quantities in the input.

The posture data acquisition unit (120) may acquire, in real time, three-dimensional (3D) body keypoint data of a subject based on two-dimensional (2D) skeleton data extracted from images of a plurality of RGB cameras (20), and may provide the same to the estimation unit (130).

The three-dimensional (3D) body keypoint data may be represented in an absolute coordinate system (a camera coordinate system/world coordinate system) or a relative coordinate system (pelvis-/thorax-based), and, for generalization according to differences in body dimensions of a subject, length normalizing (e.g., based on height or lower limb length), coordinate centering, rotation alignment (orientation alignment), etc. may be applied.

In addition, in addition to keypoint coordinates themselves, joint angles, segment direction vectors, velocity/acceleration, or skeleton graph-based feature quantities may be included, and, in preparation for non-detection/mis-detection of some keypoints, a confidence value may be input together therewith or missing-value correction (interpolation, filtering) may be performed.

The estimation unit (130) may estimate, over time, two-dimensional (2D) coordinates of a body center of pressure at each sequence length by sequentially inputting, in time order, pressure image data in units of a preset (predetermined) sequence length and three-dimensional (3D) body keypoint data in units of the preset (predetermined) sequence length to a pre-trained deep learning model.

Here, the deep learning model may be implemented to include a CNN-Bi-LSTM model having a structure in which a convolutional neural network (CNN) encoder and a bidirectional long short-term memory (Bi-LSTM) decoder are combined, a fully connected network (Fully Connected Layer), and the like.

Spatial distribution patterns (e.g., left/right and anterior/posterior load deviations, contact area changes, etc.) and temporal changes in posture data (e.g., joint angle change rates, trunk inclination trends, etc.) have different statistical characteristics.

The present disclosure is designed such that, by structurally separating and then integrating spatial feature extraction and temporal dependency learning, cross-correlation between pressure and posture is learned within the deep learning model.

As a result, it is possible to capture a predictive pattern of weight shift that is difficult to identify based only on a pressure distribution at a specific time point, and this provides a technical effect that is qualitatively distinguished from a simple weighted-average-based center-of-pressure (CoP) calculation.

In this case, the estimation unit (130) may estimate a center of pressure of the body by using body information of a subject (e.g., a height, a sex, a weight, a lean body mass, an upper limb length, a lower limb length, a waist-to-hip ratio, etc.) as additional input data to the deep learning model.

In addition, the estimation unit (130) may provide, over time, a center-of-pressure trajectory of a body based on a center of pressure of the body derived for each sequence-length unit.

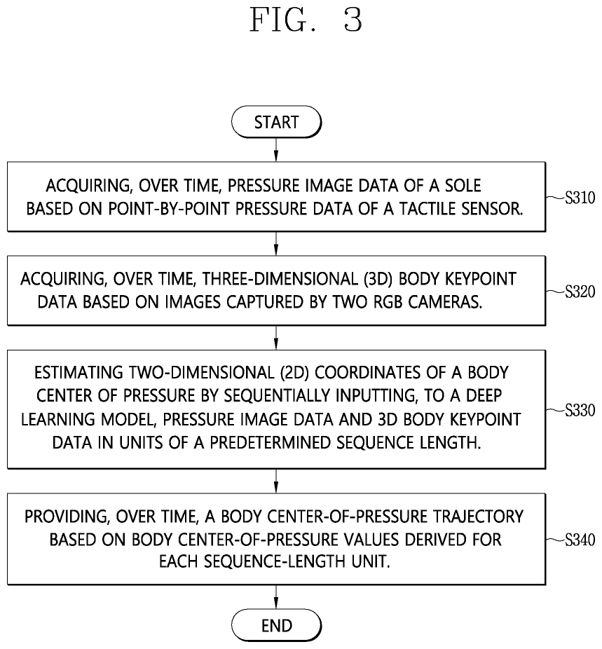

FIG. 3 is a diagram for describing a body center of pressure (CoP) trajectory estimation method using an artificial neural network according to an embodiment of the present disclosure.

First, the pressure data acquisition unit (110) may acquire, over time, pressure image data of a sole based on point-by-point pressure data measured by the tactile sensor (10) (S310).

Here, the tactile sensor (10) may measure point-by-point pressure (a pressure distribution on a two-dimensional plane) through resistance changes generated at respective intersection points between a plurality of electrode lines (e.g., copper wires) orthogonally arranged in a grid pattern on both surfaces of a thin and flexible plate-shaped piezoresistive film (e.g., a piezo film).

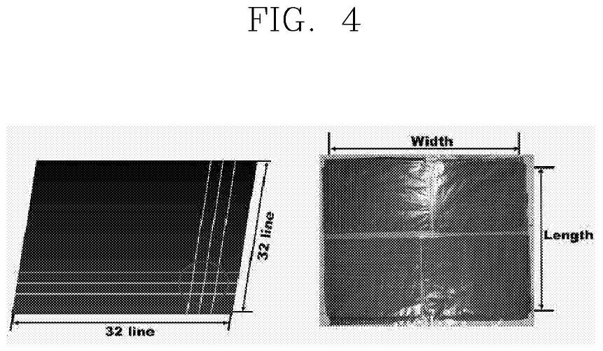

FIG. 4 is a diagram illustrating an implementation example of a tactile sensor applied to an embodiment of the present disclosure.

The left image of FIG. 4 illustrates a sensor unit body having a size of 40 cm (width)×30 cm (height) with a 32×32 grid arrangement, and the right image illustrates a tactile sensor (10) having a size of 80 cm×60 cm with a 64×64 grid arrangement using four sensor unit bodies.

Pressure data collected through four 32×32-sized sensor unit bodies may be integrated into a 64×64 size.

In this manner, it is possible to measure point-by-point (cell-by-cell) pressure (e.g., a pressure up to 14 kPa) through resistance changes generated at respective intersection points of N×M intersection points between a plurality of electrode lines orthogonally arranged in N rows and M columns (e.g., N=M=64) in the tactile sensor (10).

Of course, the basic specification of the sensor unit body may be implemented as a 64×64 grid arrangement.

Here, initial values of pressure measured for respective intersection points may differ from each other, and thus there may be a need to calibrate the initial values to 0.

In an embodiment of the present disclosure, the pressure data acquisition unit (110) may calculate an average of measured values at respective points (cells) in a state in which no weight is applied to the tactile sensor (10), and may thereafter correct the pressure values by subtracting the calculated average from pressure values at respective intersection points measured in real time.

In addition, the pressure data acquisition unit (110) may convert pressure values for respective points into N×M grayscale (0 to 255) pressure image data by applying a Min-Max scaling technique to pressure values measured for respective points to normalize observed pressure values for respective points to values between 0 and 1, and then multiplying the normalized values by 255.

For example, when N=M=64, pressure values measured for each of 64×64 intersection points may be easily converted into a grayscale pressure image composed of a total of 64×64 pixels.

In addition, as a result of multiplying, by 255, pressure values obtained by normalizing the pressure values to a range between 0 and 1, each pixel constituting the pressure image may have a pixel value (brightness value) in a range of 0 to 255.

Here, of course, in an embodiment of the present disclosure, the tactile sensor (10) is not necessarily limited to the above-described form, and any sensor or related device capable of measuring point-by-point pressure (a pressure distribution) on a two-dimensional plane may be used.

Ground-truth values of two-dimensional (2D) coordinates of a body center of pressure in a pressure image may be used for training of the deep learning model.

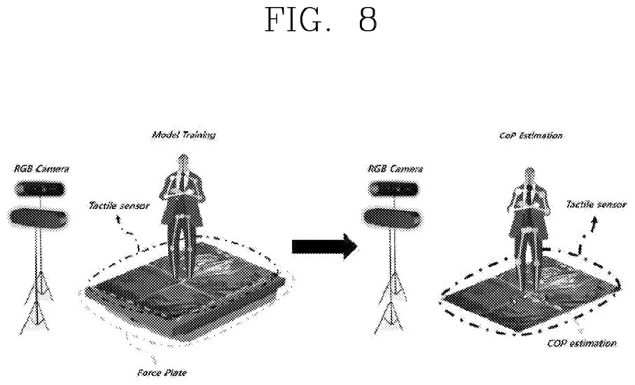

In an embodiment of the present disclosure, a ground-truth value of a body center of pressure used for training of a deep learning model may correspond to a value calculated by Equation 1 below through a weighted-average approach in consideration of a pixel value (wi) for each pixel in a pressure image, coordinates (xi, yi) of the pixel, and an actual length (L) and width (W) of the tactile sensor (10), or may correspond to a body center of pressure value actually measured by a force plate in a state in which the tactile sensor (10) is placed thereon, as in FIG. 8 described below.

A ground truth value for training is exemplified as a measured value of a force plate or a weighted-average-derived value based on pressure image data; however, the ground truth value may be configured as various reference signals depending on an application environment and data acquisition availability.

For example, a ground-truth value may be configured by combining a multi-axis force/moment sensor-based apparatus, a reference balance index provided by medical/rehabilitation equipment, or a highly reliable motion-capture-based mechanical estimate.

In addition, even when a ground-truth value is not complete (weak supervision, a noisy label), for robust learning, loss function design (e.g., a Huber loss, a confidence-weighted loss), data augmentation, or a domain adaptation technique may be applied, and this does not limit the scope of the present disclosure.

$${COP}_{X} = ( \frac{{\sum }_{i = 0}^{63} {X}_{i} {w}_{i}}{{\sum }_{i = 0}^{63} {w}_{i}} ) \times \frac{L}{64} , {COP}_{Y} = ( \frac{{\sum }_{i = 0}^{63} {y}_{i} {w}_{i}}{{\sum }_{i = 0}^{63} {w}_{i}} ) \times \frac{W}{64} \qquad Equation 1$$

Here, COP_{X}, COP_{Y} denotes two-dimensional (2D) coordinates of a body center of pressure (CoP) point.

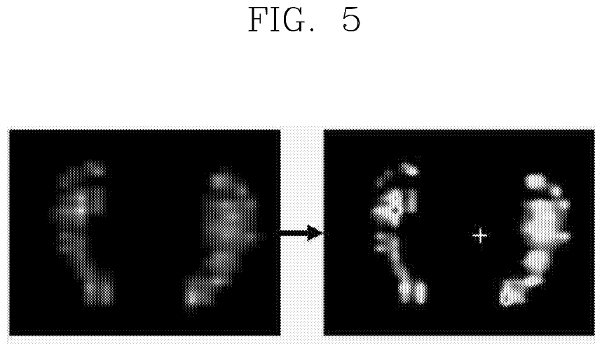

FIG. 5 is an example diagram visualizing a pressure image and a body center of pressure (CoP) point.

The left image of FIG. 5 is an example of a 64×64 grayscale image, and the right image illustrates a result obtained by converting the left image into a 628×476 colormap image using OpenCV “resize” and “applyColormap” functions and indicating, with a crosshair (“+”), a position of a body center of pressure point.

Next, the posture data acquisition unit (120) may acquire, over time, three-dimensional (3D) body keypoint data based on two-dimensional (2D) skeleton data extracted from images of a plurality of RGB cameras (S320).

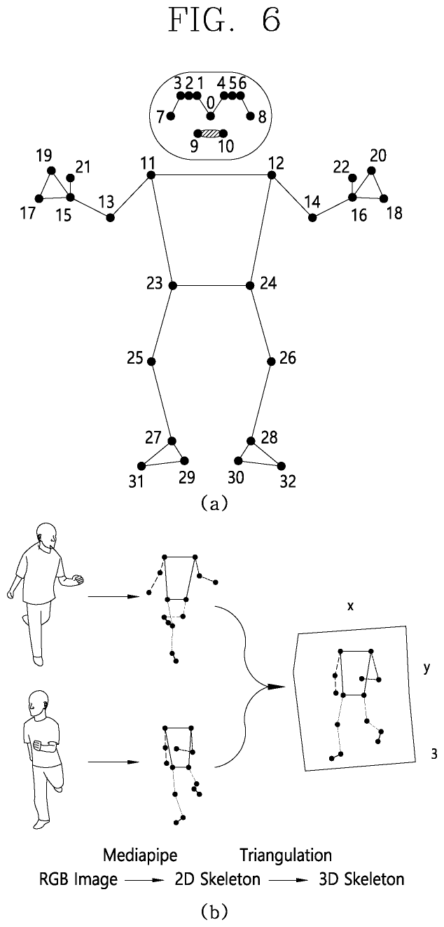

FIG. 6 is a diagram illustrating a principle of extracting a 3D skeleton of a subject using two RGB cameras in an embodiment of the present disclosure.

FIG. 6(a) defines coordinates of 33 keypoints of a body to be extracted, and FIG. 6(b) illustrates a principle of extracting a 3D skeleton using two RGB cameras.

As illustrated in FIG. 6(b), the posture data acquisition unit (120) may reconstruct, into a 3D skeleton through a triangulation technique, 2D body keypoint coordinates of a 2D skeleton extracted from images of two RGB cameras (e.g., RGB camera #1 and #2), and may acquire 3D body keypoint coordinates provided by the reconstructed 3D skeleton as three-dimensional (3D) body keypoint data.

Since the tactile sensor provides only observation information for a vertical axis, when only the tactile sensor is utilized, accuracy of estimating a body center of pressure decreases.

In an embodiment of the present disclosure, by additionally adding three-dimensional posture information provided by a 3D skeleton to sole pressure information provided by a tactile sensor, it is possible to perform more precise pressure distribution and balance analysis.

Since a plantar pressure distribution is mainly sensitive to a vertical-axis component, there may be a section in which a slight change in an upper-body tilt or a lower-limb joint is not immediately reflected in a center of pressure.

On the other hand, posture information based on a 3D skeleton directly reflects geometric changes of a trunk and limbs, but a correlation with an actual ground reaction force is indirect.

The present disclosure achieves an interaction effect in which a limitation of one modality is supplemented by another modality by combining, in a time-series manner, the two types of information in consideration of different physical origins thereof.

This effect is not automatically produced merely by adding a sensor, but is implemented by a sequence-length-unit learning structure and a fusion design.

Steps S310 and S320 may be simultaneously performed in real time, and data acquired in each step may be input to the deep learning model in units of a preset (predetermined) sequence length and used for real-time estimation of a body center of pressure.

The order of performing S310 and S320, whether to perform them in parallel, and a sequence configuration method may be flexibly changed according to system requirements.

For example, the pressure data and the posture data may be simultaneously acquired at the same time point, or may be acquired in different periods and then aligned based on timestamps, and a sequence length (Nseq) may be set not only as a fixed length but also as a variable length, or a section may be defined on an event basis (e.g., start/end of foot contact).

In addition, in order to ensure real-time performance, a movement interval (stride) of the sliding window is not limited to one frame and may be adjusted in units of multiple frames, and this may be optimized according to a trade-off between an output update period and latency.

Next, the estimation unit (130) may estimate, at each sequence length, two-dimensional (2D) coordinates (CoPx, CoPy) of a body center of pressure by sequentially inputting, to a pre-trained deep learning model, pressure image data in units of a preset (predetermined) sequence length and three-dimensional (3D) body keypoint data in units of the preset (predetermined) sequence length (S330).

In addition, the estimation unit (130) may visualize and provide, over time, a center-of-pressure trajectory of a body based on a center of pressure of the body derived for each sequence-length unit (S340).

In an embodiment of the present disclosure, the estimation unit (130) may estimate two-dimensional (2D) coordinates (CoPx, CoPy) of a center of pressure of a body by using body information of a subject as additional input data for the deep learning model.

Here, the body information may use parameters (e.g., a height, a sex, a weight, a lean body mass, an upper limb length, a lower limb length, a waist-to-hip ratio, etc.) that may affect balance information of a subject.

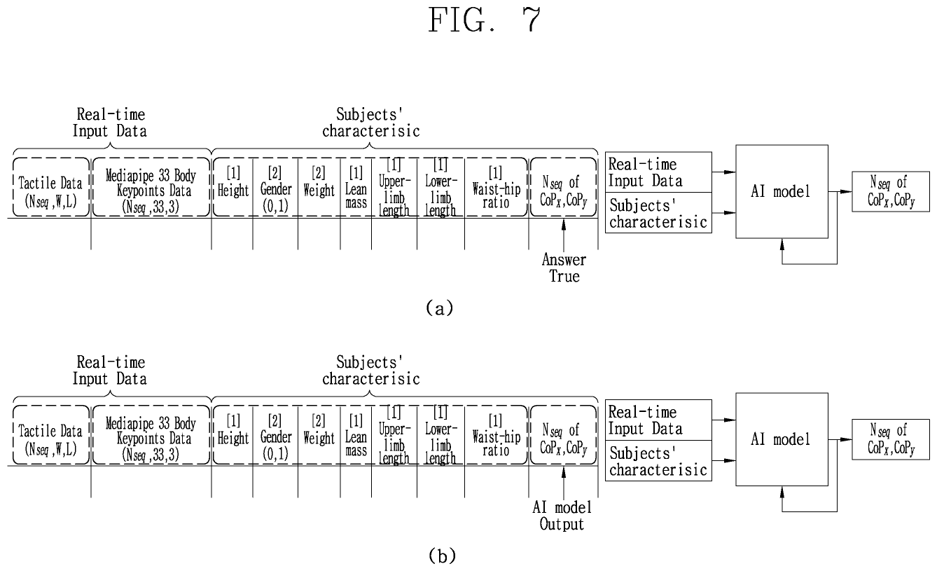

FIG. 7 is a diagram illustrating, as an example, input and output data of a deep learning model for estimating a body center of pressure according to an embodiment of the present disclosure.

FIG. 7(a) illustrates a training process of the deep learning model.

In this manner, the deep learning model may be pre-trained by using, as input data, pressure image data of a sole (Tactile Data) having a preset (predetermined) sequence length (Nseq) obtained through the tactile sensor, body posture data (33 Body keypoints Data) having the preset (predetermined) sequence length (Nseq) obtained through an RGB camera, and body information (Subject's characteristic) of a subject, and by using, as output data, a ground-truth value (Answer True) of two-dimensional (2D) coordinates of a body center of pressure in a corresponding sequence-length section.

FIG. 7(b) illustrates a process of estimating a body center of pressure through a deep learning model for which training has been completed.

The pre-trained deep learning model may estimate and provide two-dimensional (2D) coordinates of a body center of pressure for each sequence length in response to arbitrary input data given in units of a preset (predetermined) sequence length.

In this manner, according to an embodiment of the present disclosure, it is possible to estimate, for a sequence length, two-dimensional (2D) coordinates (CoPx, CoPy) of a body center of pressure by using, as inputs, Tactile Data for a predetermined sequence length collected from a tactile sensor and three-dimensional (3D) body keypoint data for the predetermined sequence length of a 3D skeleton estimated through two RGB cameras.

In addition, in this process, it is possible to increase estimation accuracy of a body center of pressure (CoP) by additionally utilizing parameters that may affect balance information of an experimenter (height, sex, weight, lean body mass, an upper limb length, a lower limb length, and a waist-to-hip ratio (waist circumference (cm)/hip circumference (cm)) information).

FIG. 8 is a diagram illustrating, as an example, a system setting for training and estimation of a deep learning model.

The left diagram of FIG. 8 illustrates a situation in which, during training of the model, pressure image data obtained by the tactile sensor, body posture data obtained by an RGB camera, and a ground-truth value of a body center of pressure obtained through a force plate are acquired, and the right diagram illustrates a situation in which, in an actual test environment, estimation of the body center of pressure is possible even without an expensive force plate.

A force plate is standard equipment for precisely measuring a ground reaction force, but has limitations of being expensive, fixed-type, and space-constrained.

The present disclosure is designed such that, in a training stage, a force plate is utilized as a reference signal, while, in an inference stage, a center of pressure (CoP) is estimated using only a tactile sensor and image-based posture information.

This is not a simple sensor substitution, but includes a technical configuration in that a mapping function learned from a reference device is transferred to a lightweight sensor environment, and thus is distinguished from a method of directly mimicking a physical function of a force plate.

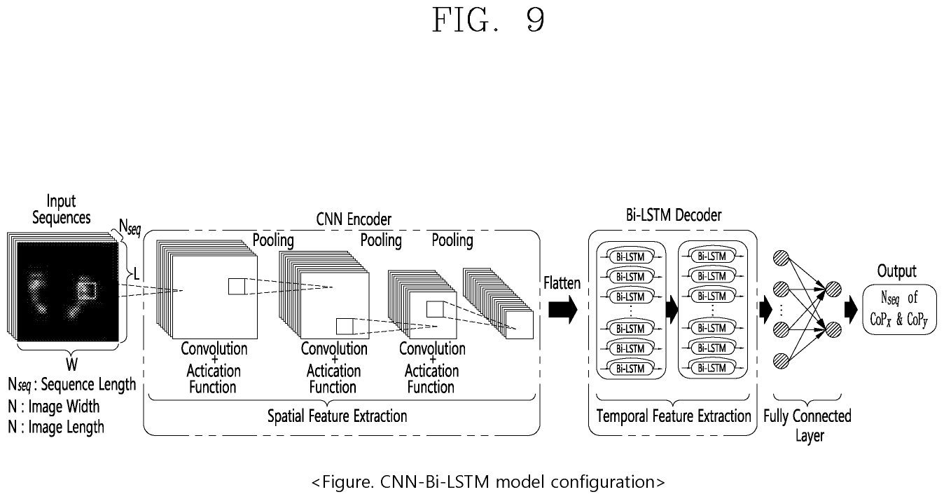

In an embodiment of the present disclosure, a deep learning model for estimating a body center of pressure (CoP) may be implemented to include a CNN-Bi-LSTM model illustrated in FIG. 9 below.

FIG. 9 is a diagram illustrating a structure of a CNN-Bi-LSTM model according to an embodiment of the present disclosure.

As illustrated in FIG. 9, the CNN-Bi-LSTM model may have a structure in which a convolutional neural network (CNN) encoder configured to apply a sliding window to pressure image data having a preset (predetermined) sequence length (Nseq length) to extract a spatial feature within the window and a bidirectional long short-term memory (Bi-LSTM) decoder configured to bidirectionally learn a temporal dependency with respect to the extracted spatial feature are combined.

A conventional CNN-LSTM model has a limitation in that an LSTM processes data only in one direction, but a CNN-Bi-LSTM model combines a bidirectional long short-term memory (Bi-LSTM) decoder with a convolutional neural network (CNN) encoder to simultaneously learn bidirectional information, thereby enabling more effective learning of a temporal dependency.

The CNN-Bi-LSTM model may first extract a spatial feature of a pressure image through a convolutional neural network (CNN) encoder, and then may bidirectionally learn a temporal dependency through a bidirectional long short-term memory (Bi-LSTM) decoder.

In addition, a sliding window technique is applied in order to efficiently learn a temporal pattern of pressure data and minimize loss during real-time time-series data analysis.

A sliding window is a method of learning by dividing data into windows having a predetermined length, and is useful for minimizing loss of continuous time-series information, such as pressure data, when processing sequence data.

Each sequence may be designed to move by one frame through a sliding window and may be used as an input to the model.

Through this, it is possible to estimate a center-of-pressure (CoP) trajectory in real time.

Hereinafter, an experimental method and experimental results for estimating a body CoP through a technique of the present disclosure will be described.

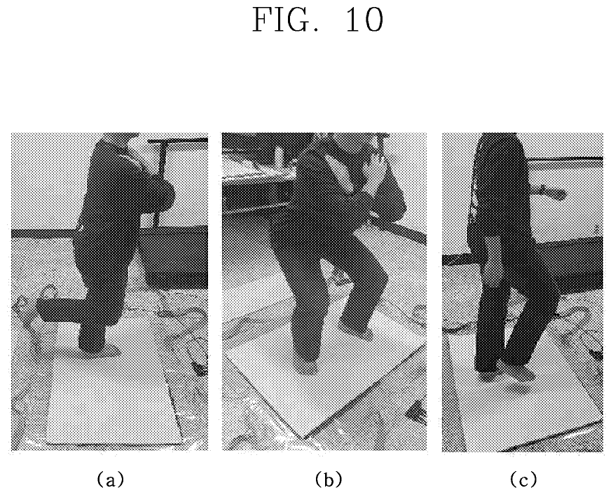

FIG. 10 is a diagram illustrating an experimental environment and a protocol for a performance test of a technique of the present disclosure.

First, as illustrated in FIG. 10, in order to estimate a body CoP, three static and dynamic balance postures were performed by an experimental participant.

The experiment participants consisted of a total of ten healthy adult males (age: 24±1.15 years, height: 1.74±0.03 m, weight: 71.54±6.76 kg).

The protocol is largely classified into static balance postures (one-leg standing) and dynamic balance postures (squat and walking in place), and corresponds to postures commonly performed in balance evaluation.

For a one-leg standing posture, the experimental participant was asked to stand on one dominant leg and maintain balance.

For a squat posture, a participant was requested to perform the motion at a constant rate of one every 3 seconds, and for an in-place walking motion, the participant was requested to perform the motion at a constant rate at a comfortable speed.

Data collection was performed for 80 seconds per protocol, and each motion was measured by repeating it twice.

A rest time of 30 seconds was provided between the same motions, and a rest time of 60 seconds was provided between different motions such that subjects were able to sufficiently recover.

The experiment participants performed the experiment in a state in which, as illustrated in the figure below, a silicone mat having a thickness of 10 mm was placed on the tactile sensor.

A mat was applied to increase convenience for the experiment participant and to prevent long-term degradation in sensor performance.

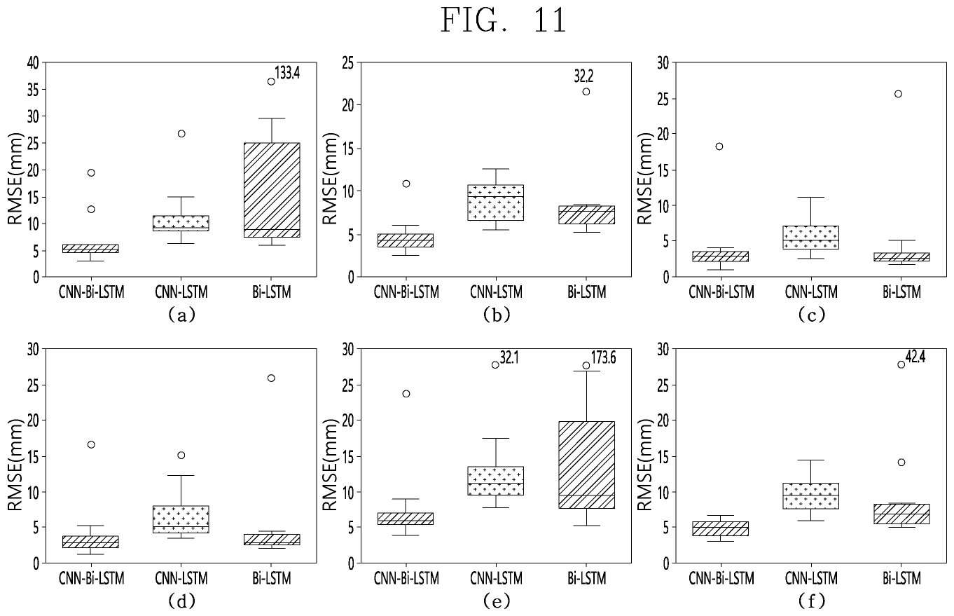

FIG. 11 is a diagram illustrating performance of estimating a body center of pressure (CoP) through a CNN-Bi-LSTM model.

Training was performed for each of a Bi-LSTM, a CNN-LSTM, and a CNN-Bi-LSTM model using the same data, and it can be confirmed through FIG. 11 that the CNN-Bi-LSTM model exhibited superior estimation performance of a body center of pressure (CoP) for both static and dynamic postures compared to the other two conventional models.

The experiment described above is an example performed under specific participant groups and posture conditions; however, the technical spirit of the present disclosure is also applicable even when a participant's age, sex, body type, posture type, sensor resolution, and camera arrangement differ.

In particular, an improvement in performance in a dynamic posture may be understood to be attributable to a spatiotemporal fusion learning structure, and this may be extended to various balance tasks and rehabilitation scenarios.

Accordingly, an advantageous effect of the present disclosure is not limited to a specific experimental protocol.

In this manner, according to the present disclosure, it is possible to accurately estimate a body center of pressure (CoP) through a high-resolution pressure image acquirable through a tactile sensor and three-dimensional (3D) posture information acquirable through a low-cost RGB camera, even without an expensive force plate.

In addition, the present disclosure can more accurately estimate a body center of pressure (CoP) trajectory by overcoming a limitation of a conventional tactile sensor capable of providing only vertical-axis tactile information and by integrally utilizing, as an input to the deep learning model, three-dimensional (3D) body keypoint data based on a 3D skeleton.

In addition, the present disclosure may be implemented as a sensor-based system and thus may be easily installed and used not only in a hospital but also in a home and a rehabilitation center, and may provide real-time data processing and visualization.

The present disclosure has been described with reference to embodiments illustrated in the drawings; however, these are merely examples, and a person having ordinary skill in the art will understand that various modifications and other equivalent embodiments may be possible therefrom.

Therefore, the true technical scope of protection of the present disclosure should be defined by the technical spirit of the appended claims.

1. An apparatus for estimating a trajectory of a body center of pressure using an artificial neural network, comprising:
a pressure data acquisition unit configured to acquire, over time, pressure image data of a sole based on point-by-point pressure data of a plate-type tactile sensor;
a posture data acquisition unit configured to acquire, over time, three-dimensional (3D) body keypoint data based on two-dimensional (2D) skeleton data extracted from images of a plurality of RGB cameras; and
an estimation unit configured to estimate, at each sequence length, two-dimensional (2D) coordinates of the body center of pressure by sequentially inputting, in time order, pressure image data and 3D body keypoint data in units of a predetermined sequence length to a pre-trained deep learning model.

2. The apparatus of claim 1, wherein the estimation unit is configured to provide a center-of-pressure trajectory of a body over time based on a center of pressure of the body derived for each sequence-length unit.

3. The apparatus of claim 1, wherein the estimation unit estimates a center of pressure of the body by additionally inputting, to the deep learning model, body information including at least one of a height, a sex, a weight, a lean body mass, an upper limb length, a lower limb length, and a waist-to-hip ratio of a subject.

4. The apparatus of claim 1, wherein the deep learning model is implemented to include a CNN-Bi-LSTM model having a structure in which a convolutional neural network (CNN) encoder configured to apply a sliding window to pressure image data having the preset sequence length to extract a spatial feature within the window and a bidirectional long short-term memory (Bi-LSTM) decoder configured to bidirectionally learn a temporal dependency with respect to the spatial feature are combined.

5. The apparatus of claim 1, wherein the tactile sensor measures point-by-point pressure through resistance changes generated at respective intersection points between a plurality of electrode lines orthogonally arranged in a grid pattern on both surfaces of a plate-shaped piezoresistive film.

6. The apparatus of claim 1, wherein the pressure data acquisition unit acquires N×M grayscale pressure image data in which numerical values, obtained by normalizing, to values within a set range, pressure values measured for each intersection point between a plurality of electrode lines orthogonally arranged in N rows and M columns in the tactile sensor, are used as pixel values on respective two-dimensional coordinates.

7. The apparatus of claim 1, wherein the posture data acquisition unit obtains 3D body keypoint coordinates of a 3D skeleton as the 3D body keypoint data based on a result of reconstructing, into the 3D skeleton through a triangulation technique, 2D body keypoint coordinates of a 2D skeleton extracted from images of two RGB cameras.

8. A body center of pressure (CoP) trajectory estimation method performed by an apparatus for estimating a trajectory of a body center of pressure using an artificial neural network, the method comprising: acquiring, over time, pressure image data of a sole based on point-by-point pressure data of a plate-type tactile sensor; acquiring, over time, three-dimensional (3D) body keypoint data based on two-dimensional (2D) skeleton data extracted from images of a plurality of RGB cameras; and estimating, at each sequence length, two-dimensional (2D) coordinates of a center of pressure of a body by sequentially inputting, to a pre-trained deep learning model, pressure image data and 3D body keypoint data in units of a preset (predetermined) sequence length.

9. The method of claim 8, further comprising providing, over time, a center-of-pressure trajectory of a body based on a center of pressure of the body derived for each sequence-length unit.

10. The method of claim 8, wherein the step of estimating the center of pressure of the body comprises estimating the center of pressure of the body by additionally inputting, to the deep learning model, body information including at least one of a height, a sex, a weight, a lean body mass, an upper limb length, a lower limb length, and a waist-to-hip ratio of a subject.

11. The method of claim 8, wherein the deep learning model is implemented to include a CNN-Bi-LSTM model having a structure in which a convolutional neural network (CNN) encoder configured to apply a sliding window to pressure image data having the preset (predetermined) sequence length to extract a spatial feature within the window and a bidirectional long short-term memory (Bi-LSTM) decoder configured to bidirectionally learn a temporal dependency with respect to the spatial feature are combined.

12. The method of claim 8, wherein the tactile sensor measures point-by-point pressure through resistance changes generated at respective intersection points between a plurality of electrode lines orthogonally arranged in a grid pattern on both surfaces of a plate-shaped piezoresistive film.

13. The method of claim 8, wherein the acquiring, over time, the pressure image data comprises acquiring N×M grayscale pressure image data in which numerical values, obtained by normalizing, to values within a set range, pressure values measured for each of intersection points between a plurality of electrode lines orthogonally arranged in N rows and M columns in the tactile sensor, are used as pixel values on respective two-dimensional coordinates.

14. The method of claim 8, wherein the step of acquiring, over time, the three-dimensional (3D) body keypoint data comprises acquiring, as the three-dimensional (3D) body keypoint data, 3D body keypoint coordinates of a 3D skeleton derived by reconstructing, into the 3D skeleton through a triangulation technique, 2D body keypoint coordinates of a 2D skeleton extracted from images of two RGB cameras.
